(19) United States
(12) Patent Application Publication
(10) Pub. No.: US 2015/0141910 A1
(43) Pub. Date: May 21, 2015

(54) MICRONEEDLES FOR THERAPEUTIC AGENT DELIVERY WITH IMPROVED MECHANICAL PROPERTIES

(71) Applicant: University Medical Pharmaceuticals Corporation, Irvine, CA (US)

(72) Inventors: Raymond J. Francis, Laguna Beach, CA (US); Gregory Hunt, Corona Del Mar, CA (US); Ryan F. Donnelly, Belfast (GB)

(21) Appl. No.: 14/542,369

(22) Filed: Nov. 14, 2014

Related U.S. Application Data
(60) Provisional application No. 61/904,421, filed on Nov. 14, 2013.

(51) Int. Cl. A61M 37/00; A61L 31/04; C08L 39/06

(52) U.S. Cl. CPC A61M 37/0015; C08L 39/06; A61L 31/041; A61M 2037/0023; A61M 2037/0046; A61M 2037/0053
USPC 604/46; 524/27; 264/255

(57) ABSTRACT

Disclosed herein are systems and methods relating to microneedles, including a first element including an array of microprojections and a second element including a supportive substrate upon which the microprojections are formed perpendicular to the substrate surface.

### PRIORITY CLAIM

This application claims the benefit under 35 U.S.C. §119(e) of U.S. Prov. Pat. App. No. 61/904,421 filed on Nov. 14, 2013, which is hereby incorporated by reference in its entirety.

### BACKGROUND

Various methods to deliver a therapeutic agent into the skin can be used, including via injection, topical agents, and iontophoresis for example. Injections can be painful, even with anesthetics, and many patients have an aversion to needles. It may be difficult for topical agents to penetrate the stratum corneum into the deeper layers of the skin. Microneedles have an advantage of potentially penetrating the stratum corneum, without the discomfort of conventional needles, and can be self-administered. However, improved microneedles are needed that can effectively deliver the therapeutic agent into the desired target anatomical location.

### SUMMARY

The devices and methods herein provide pathways for introducing agents into the skin without the discomfort of conventional needles. In several embodiments, the device comprises an array of microneedles that project from a face of a substrate. In some embodiments, the device is reusable and has circuitry that enables iontophoresis to drive therapeutic agents through the microneedle and into the skin. In several embodiments, the microneedle array is a disposable patch that couples with a reusable iontophoresis component of the device. In some embodiments, the device includes an adhesive layer allowing skin retention of the device. In at least one embodiment, the device includes a protective water-insoluble occlusive layer.

In some embodiments, the microneedles extend from a substrate made from the same material as the microneedles. In several embodiments the microneedles extend from a substrate made of a material having a different composition than the material used to make the microneedles. In some embodiments, the microneedles are made of a material containing hyaluronic acid or derivatives thereof. In at least one embodiment, the microneedles are made of a material that includes hyaluronic acid having an average molecular weight in the range of 100,000 Da to 2,000,000 Da. In some embodiments, the microneedles are made of a material that contains hyaluronic acid, or derivative thereof, that is crosslinked with a cationic agent. In at least one embodiment, the microneedles comprise hyaluronic acid, or derivative thereof, that is crosslinked with chitosan or a derivative thereof. In some embodiments, the microneedles are made of a material that contains polyvinylpyrrolidone, polyvinylalcohol, a cellulose derivative, or other water soluble biocompatible polymer. In some embodiments, the microneedles are made of a material that contains polyvinylpyrrolidone having an average molecular weight between about 20 kDa and about 100 kDa. In some embodiments, the substrate is made of a material that contains polyvinylpyrrolidone having an average molecular weight between about 20 kDa and about 100 kDa. In some embodiments, the substrate is made of a material comprising between about 20% and about 50% polyvinylalcohol.

In some embodiments, the microneedles are made of a material configured to swell in skin interstitial fluid upon skin insertion. In at least one embodiment, the microneedles dissolve in skin interstitial fluid upon skin insertion. In several embodiments, the substrate is water soluble and dissolves upon skin insertion of the microneedles. In at least one embodiment, the substrate is water soluble and dissolves within about 15 minutes to about 6 hours of skin insertion of the microneedles.

In several embodiments, the device is configured so that single or repeated use of the device causes a noticeable increase in skin volume at the site of application. In some embodiments, single or repeated use of the device causes a noticeable reduction in the appearance of wrinkles, fine lines, stretch marks, or acne scars at the site of application.

In some embodiments, the device includes electrodes and a source of direct or alternating current. In several embodiments, the device is configured to apply an electrical current. In at least one embodiment, the application of an electrical current enhances the rate of hyaluronic acid deposition into the skin. In some embodiments, application of electrical current accelerate microneedle dissolution or swelling in the skin. In some embodiments, application of electrical current accelerates dissolution of the supportive substrate.

In several embodiments, the microneedles are substantially perpendicular to the substrate. In some embodiments, the microneedles have a height in the range of about 100 μm to about 1000 μm. In some embodiments, the microneedles have an interspacing in the range of about 50 μm to about 1000 μm. In several embodiments, the density of the microneedles on the substrate is in the range of about 50 to about 5000 microneedles per cm². In some embodiments, the microneedles are conical in shape. In some embodiments, the microneedles are cylindrical in shape. In at least one embodiment, the microneedles are pyramidal in shape.

In several embodiments, the microneedle array is formed by casting. In at least one embodiment, the microneedle array is formed by a two-stage casting process wherein the microneedles are formed in the first stage and the substrate is formed in the second stage.

### BRIEF DESCRIPTION OF THE DRAWINGS

A more complete appreciation of the subject matter of this application and the various advantages thereof can be realized by reference to the following detailed description, in which reference is made to the accompanying drawings.

FIG. 1 shows one embodiment of the device;

FIG. 2 is a cross-sectional view of an embodiment of the device inserted into the skin;

FIG. 3 shows a schematic of microneedle swelling and substrate dislocation from the microneedles after insertion of the microneedles into the skin.

FIG. 4 is a gmph showing microneedle swelling over time for the experiment described in Example 3 herein.

FIG. 5 is a table showing microneedle swelling over time for the experiment described in Example 4 herein.

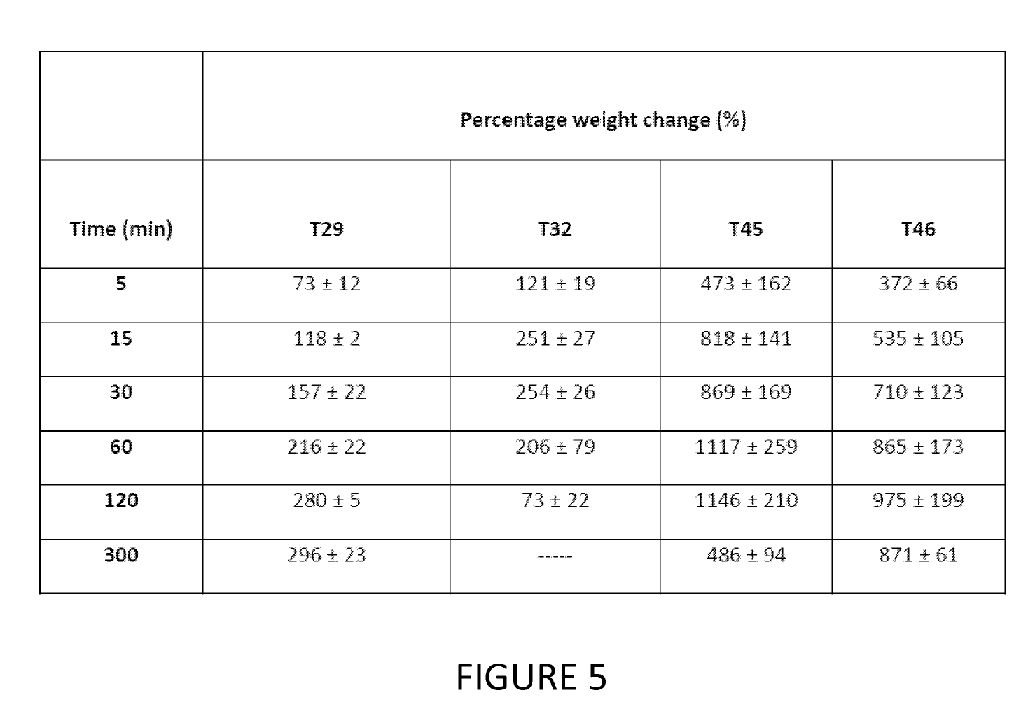

### DETAILED DESCRIPTION

Iontophoresis has been shown to deliver active ingredients transdermally in a much more efficacious manner than simple topical applications of cosmetic ingredients. For the specific case of delivery of hyaluronic acid for wrinkle reduction, this performance improvement can be 20-fold or more compared to a topical approach.

Some devices for iontophoretic delivery of active compounds include a hand-held device that glides over topical ingredients applied to the skin (e.g. Nu-Skin). Such devices can provide uneven results. For example, the amount of active ingredients applied to the skin varies widely, the application time is uncontrolled, and the application time is not consistent for different areas.

There are also devices connected to disposable patches with 2 or more built-in electrodes (e.g. Vytaris, Dharma). Such devices that connect to patches with multiple electrodes can have drawbacks in that they add complexity and cost to the single-use patches, limit the delivery of the actives to the size of one of the 2 electrodes, and typically delivers active ingredients of only a single polarity (with the other electrode containing just saline solution to complete the circuit).

Other devices include patches with integral electronics (e.g. Empi, Isis). Devices with integral electronics can be too expensive for use with cosmetics since the electronics are single use only.

Also, devices connected to disposable patches via a wiring harness are known (e.g. WrinkleMD). Such devices can use 2 symmetrical electrodes with the device driving a cycle with alternating polarity. This architecture has the benefit of simple patch construction, uniform coverage, ability to deliver active ingredients of both polarities, and programmable cycle with defined ramps, ON times, and dwell times that can be tuned to increase efficacy and improve user comfort. Improvements and wireless embodiments of such systems are disclosed herein.

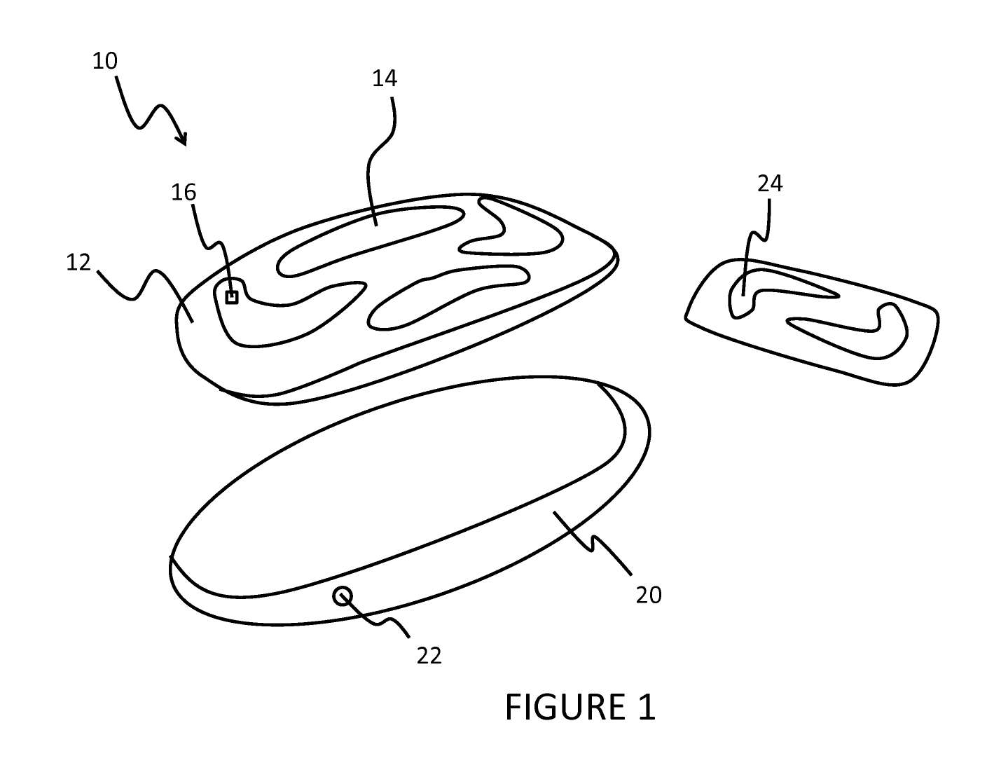

FIG. 1 discloses a cosmetic agent delivery system 10 for delivery of a cosmetic agent into the skin, according to some embodiments of the invention. The system 10 can include a housing 12, e.g., a travel case that holds one, two, or more masks 14 (this example includes a brow, lip and eye masks). The system 10, or each mask 14 can include a power source 16, such as a rechargeable battery (or equivalent energy storage device such as a capacitor). The power source 16 can store sufficient energy for multiple uses. The system 10 can include a docking station 20 to re-charge the masks 14 prior to use, and in some cases includes a charge status indicator 22, such as an LED indicator for example.

The masks 14 can be configured for multiple uses, and be configured as pre-contoured geometries tailored to application to different body parts depending on the desired clinical result (e.g. brow, eyes, lip). The masks 14 can be made of flexible, low-durometer materials, such as plastics, silicone, polymers, etc. that conform to a variety of face shapes. Each mask 14 can include, for example, integral electrodes (e.g., one, two, or more electrodes; electrode pattern tailored to application), integral control electronics, and/or logic to control the delivered dose (current and time). A controller (not shown) can include programmable polarity, cycle time, dwell time, etc. In some embodiments, the system 10 is configured to reverse polarity at least 2, 3, 4, 5, 6, 7, 8, 9, or 10 times to provide more even distribution of active ingredients, which can be advantageous when applying bilaterally symmetric masks and patches, such as on both sides of the face for example. In some embodiments, in addition to or in place of components configured to perform iontophoresis, the system 10 could include one, two, or more modalities to synergistically increase transdermal penetration of therapeutic agents such as those disclosed elsewhere herein.

In some embodiments, the mask 14 can also include indicia, such as visual/audible user feedback (e.g. IN USE, DONE), and wired or wireless connectivity (e.g., e.g., Bluetooth® radio technology, communication protocols described in IEEE 802.11 (including any IEEE 802.11 revisions), Cellular technology (such as GSM, CDMA, UMTS, EVDO, WiMAX, or LTE), or Zigbee® technology, among other possibilities). In some embodiments, the connectivity allows the mask 14 to communicate with a remote device, such as a desktop or laptop computer, tablet, or smartphone application for example. The remote device can include one or more applications able to display one or more of the following: the total target dose for each mask in use; the dose delivered at any given time during use; the date/time of each use; the total number of uses for each mask type; reminders to replenish the single-use patches; and links to order additional devices and single-use patches.

In some embodiments, the electrode pattern is molded directly into the masks 14 with a disposable component defining one, two, or more layers affixed to the mask 14. In this case, advantageously no electrode is required as part of the disposable, and the mask component can be reused.

The system 10 can also include patches 24, e.g., single-use patches with contoured geometries tailored to application to different sites on one's face or body (e.g. brow, eyes, lip). In some embodiments, the contoured geometries of the patches 24 are substantially complementary to that of the masks 14. The patches 24 can include one, two, or more active ingredients tailored to each body part and/or skin type. For example, the patches 24 can include a hydrogel (or equivalent) with active and/or passive ingredients in a predetermined pattern to match with electrodes on the corresponding mask 14. In some embodiments, the hydrogel is uniform and has a sufficiently high lateral resistivity such that the current does not short-circuit through the hydrogel and instead goes through the skin. In some embodiments, the hydrogel can have adhesive material(s) on first and/or second surfaces (e.g., both sides) of the patches—one to adhere to the mask 14 and one to adhere to the skin. These adhesives may be similar or may have greater adhesive properties on one side with respect to another side as appropriate for user convenience/comfort. In some embodiments, the patch 24 can include an active adhesive film instead of or in addition to a hydrogel.

Hydrogels have three dimensional network structure of polymer chains holding significant amount of water. The water holding capacity of a hydrogel depends upon the basic polymer network structure, other ingredients and the production process. Synthetic and natural polymers along with other chemical ingredients have been used for making hydrogels.

Hydrogel materials can include, for example, one or more of polyvinyl pyrrolidone, vinyl pyrrolidone, acrylamide, poly vinyl alcohol, polyethylene oxide, gelatin, agar-agar, a glycosaminoglycan polymer, a hyaluronic acid-based polymer, and the like. In some embodiments, the active ingredients may include hyaluronic acid, stressed yeast cell lysate, yeast cell derivative, and cross-linked synthetically derived protein. As used herein, hyaluronic acid (HA) can refer to any of its hyaluronate salts, and includes, but is not limited to, sodium hyaluronate (NaHA), potassium hyaluronate, magnesium hyaluronate, calcium hyaluronate, and combinations thereof. In some embodiments, the concentration of HA in the compositions described herein is preferably at least 10 mg/mL and up to about 40 mg/mL. For example, the concentration of HA in some of the compositions is in a range between about 20 mg/mL and about 30 mg/mL. In some embodiments, the HA comprises between about 0.1% and about 15% by weight of the entire composition.

Disclosed herein are embodiments of systems and methods for the delivery of active ingredients into the skin using iontophoresis and/or other modalities described elsewhere herein, that can include one, two, or more microneedles operably connected to a patch 24 containing active ingredients. The systems and methods can advantageously be able to deliver a controlled dose of active ingredients using iontophoresis. The systems can include single-use substrates, e.g., patches 24, with one, two, or more active ingredients that are reversibly mateable to a mask 14. The mask 14 can include, for example, control electronics, and one, two, or more electrodes that are arranged to deliver the active ingredients in a defined pattern. In some embodiments, the microneedle patches can be utilized alone, e.g., in the absence of another modality such as iontophoresis.

In some embodiments, the system 10 could include one, two, or more modalities to synergistically increase transdermal penetration of therapeutic agents such as those disclosed herein. Not to be limited by theory, but some modalities increase permeability of dermatologic preparations through the stratum corneum layer. Such permeability-enhancing modalities could involve, but are not limited to one, two, or more of mechanical, chemical, thermal, and electromagnetic modalities, including sonophoresis, iontophoresis, RF, laser, microwave, and pulsing electromagnetic fields, for example. In some embodiments, the permeability-enhancing modality involves applying a chemical peel to the skin, such as, for example, glycolic or salicylic acid, or a retinoid. While chemical solvents can be used with positive effect, in some embodiments they can undesirably dissolve, denature, or otherwise alter the dermatologic preparation. In some embodiments, the permeability-enhancing modality involves applying heat to the skin. In some embodiments, iontophoresis is employed. In some embodiments, iontophoretic delivery of therapeutic agents into the skin can be as described, or modified from U.S. Pub. No. 2011/0190724 A1 to Francis et al., which is hereby incorporated by reference in its entirety. In some embodiments, the preparation can be administered under occlusion to synergistically increase penetration, in other words, to trap the preparation against the skin to increase penetration and effect.

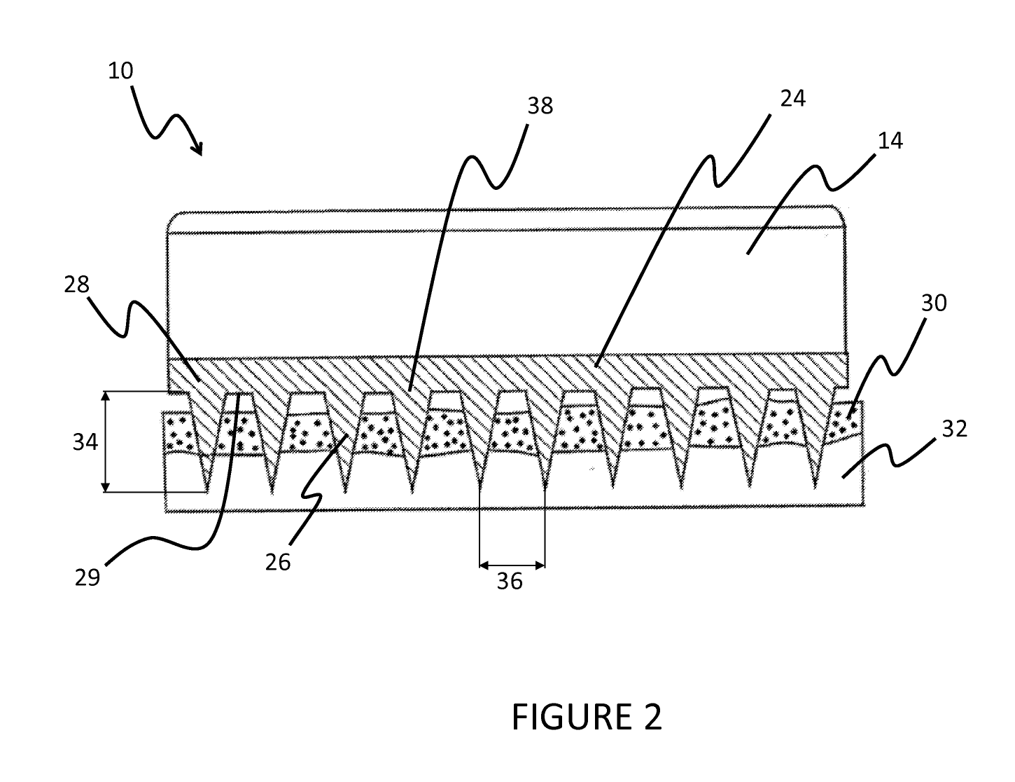

Referring to FIG. 2, some embodiments of the patch 24 include a microneedle array. The patch 24 including microneedles 26 can, in some cases, have the following attributes: (1) the strength to withstand insertion into the skin surface layer and/or stratum corneum; (2) the fineness and flexibility to cause no pain or bleeding in the skin surface layer and/or stratum corneum at the insertion site of the microneedles, and/or (3) solubility or biodegradability in the body of the microneedle portions under the skin. Not to be limited by theory, but a patch 24 containing microneedles having one, two, or more active ingredients, such as hyaluronic acid for example, has surprisingly and unexpectedly showed skin penetration and clinical results such as wrinkle reduction, either alone or in combination with the system components and iontophoresis with parameters as described elsewhere herein.

The inventors have discovered formulations as described herein that surprisingly have been able to form intact microprojections configured to deliver therapeutic amounts of agents into the skin. Microneedle manufacturing utilizing inappropriate materials, concentrations, molecular weights, and other parameters can result in problems including non-formation, mal-formation, or overly brittle microneedles that are unable to penetrate into the skin without fracturing.

In some embodiments, the microneedle array includes any number of microneedles, such as about 10 to about 500 microneedles, about 50 to about 250 microneedles, or about 50, 100, 150, 200, 250, 300, 350, 400, 450, or 500 microneedles in some embodiments.

FIG. 2 is a schematic elevational view in partial cross-section of a cross-linked array of microneedles 26 forming part of a transdermal delivery system 10 for the delivery of a therapeutic agent. Shown is the epidermis 30 and stratum corneum 32 of a patient's skin; microneedles 26 extending distally from one surface of patch 24; and the mask 14 reversibly mated to the patch 24, e.g., via an adhesive.

In some embodiments, the microneedles 26 are formed on the surface of a substrate 28, and are made of a material containing hyaluronic acid or another active ingredient. The microneedles 26 could take any appropriate cross-section, such as triangular, rectangular, circular, oval, or elliptical for example, and/or take the form of cones, rods, pyramids and/or cylinders. As such, the microneedles may have the same diameter at the tip as at the base or may taper in diameter in the direction base to tip. The microneedles 26 may have at least one sharp edge and may be sharp at the tips. The microneedles 26 may be solid, have a hollow bore down at least one longitudinal axis at an angle to the substrate 28 and extending to the first side 29 of the substrate 28, they may be porous, or may have at least one channel running down at least one outer surface from tip to substrate 28.

As noted above, in order to be of use in transdermal delivery, arrays of microneedles 26 can be capable of creating openings in the stratum corneum 30 barrier through which beneficial substances can move. Thus, the force of insertion is less than the force required to fracture the microneedles 26. In some embodiments, the microneedles 26 do not fracture when a pressure of insertion of less than about 10, 0.5, 9, 8.5, 8, 7.5, 7, 6.5, 6, 5.5, 5.0, 4.5, 4, or 3.5 N/cm², for example less than 3.0 N/cm², such as less than 0.5 N/cm² is exerted on the microneedles 26 along their length. In some embodiments, the microneedles can be configured to bend but not break upon application of a defined force, such as about or no more than about 50, 45, 40, 35, 30, 25, 20, or 15 N/array for a defined time period, such as about 30 seconds, 45 seconds, or 60 seconds for example.

In some embodiments, the modulus is a material property which indicates a material's resistance to deformation, or stiffness. Tough describes the energy absorbed, or work done, by the material to resist deformation. A material which absorbs a high degree of energy before failure is described as ductile whilst one which absorbs little. In some embodiments, one which absorbs a relatively greater amount of energy can be preferred. In some embodiments, the microneedles can have a modulus of between about 0.10 and about 0.40 MPa, between about 0.15 and about 0.30 MPa, or about, at least about, or no more than about 0.20, 0.22, 0.24, 0.26, 0.28, or 0.30 MPa. In some embodiments, the microneedles can have a toughness of between about 170 and about 250 Nmm, between about 180 and about 240 Nmm, or about or at least about 170, 180, 190, 200, 210, 220, 230, 240, 250, or more Nmm.

A microneedle 26 can be any suitable size and shape for use in an array to puncture the stratum corneum 30. The microneedles 26 of the array can be configured to pierce and optionally cross the stratum corneum 30. The height 34 of the microneedles 26 can be altered so as to allow penetration into the upper epidermis 32, as far as the deep epidermis or even the upper dermis, but not allowing penetration deep enough into the skin to cause bleeding. In one embodiment, the microneedles 26 are conical in shape with a circular base which tapers to a point at a height of the microneedles above the base. In some embodiments,

In some embodiments, the microneedles 26 have a root diameter of 120 to 400 μm, a tip diameter of 5 to 100 μm, a height 34 of 100 to 5000 μm, and the pitch (the distance from tip to tip) between adjacent microneedles is 100 to 1800 μm. In some embodiments, the microneedles 26 have a height 34 of 100 to 1600 μm or 100 to 1000 μm; a height 34 of more than 1000 μm but not more than 5000 μm, or more than 1000 μm but not more than 3000 μm; or a height 34 of more than 1600 μm but not more than 5000 μm, or more than 1600 μm but not more than 3000 μm. In some embodiments, the microneedles can have a height of about 200, 250, 300, 350, 400, 450, 500, 550, 600, 650, 700, 750, 800, 850, 900, 950, or 1000 μm or ranges including any two of the foregoing. In some embodiments, the microneedle arrays have a millimeter-order height 34 as above mentioned, but a micrometer-order fineness (the root diameter and the tip diameter of needle).

In some embodiments of the microneedle array, the microneedles 26 can be in the range of 1 μm to 3000 μm in height 34. For example, the microneedles 26 can have heights 34 in the range of about 50 μm to 400 μm, for example 50 to 100 μm. Suitably, in embodiments of the arrays of the invention, microneedles 26 can have a width, e.g. diameter in the case of microneedles of circular cross-section diameter of 1-500 μm at their base. In one embodiment microneedles 26 can have a diameter in the range 50-300 μm, for example 100-200 μm. In another embodiment, the microneedles 26 can be of a diameter in the range of 1 μm to 50 μm, for example in the range 20-50 μm.

The apical separation distance 36 between each of the individual microneedles 26 in an array can be modified to ensure penetration of the skin while having a sufficiently small separation distance to provide high transdermal transport rates. In embodiments of the device the range of apical separation distances 36 between microneedles 26 can be in the in the range 50-1000 μm, such as 100-300 μm, for example 100-200 μm. This allows a compromise to be achieved between efficient penetration of the stratum corneum 30 and enhanced delivery of therapeutic active agents or passage of interstitial fluid or components thereof.

In some embodiments, the substrate (e.g., a baseplate) can include one, two, or more water-soluble materials, such as PVP or other polymers as disclosed herein. The substrate can also include an adhesive in some embodiments to maintain proper positioning of the device. The microneedles can also be configured to detach from the substrate in some embodiments.

In some embodiments, the polymers of the microneedles 26 are crosslinked, either physically, chemically or both. The microneedle array can comprise groups of microneedles 26 wherein a first group comprises at least one different cross-linker to at least a second group.

In some embodiments the microneedles 26 may not be crosslinked and will dissolve following an initial swelling phase upon puncturing the stratum corneum 30 and coming into contact with skin moisture. In this case, the therapeutic active agents can be released into the skin at a rate determined by the rate of dissolution of the microneedles 26.

The rate of dissolution of particular microneedles 26 is dependent on their physicochemical properties which can be tailored to suit a given application or desired rate of drug release. Relatively slow dissolution times can, in some cases, advantageously enable prolonged retention of the active compound. In some embodiments, the microneedles can have a dissolution time of about or at least about 60, 75, 90, 105, 120, 135, 150, 165, 180, 195, 210, 225, 240, 300, 360, 420, 480, 600, 720, or more minutes, or 6, 8, 10, 12, 14, 16, 18, 20, 22, 24, 28, 32, 36, 40, 44, 48 hours, or more.

In some embodiments, microneedles absorb interstitial fluids, e.g., fluids within the skin in order to increase volume and provide an improved aesthetic appearance, e.g., to eliminate or improve wrinkles for example. In some embodiments, the microneedles can, after insertion into the stratum corneum, have a maximal increase in weight (e.g., by the absorption of interstitial fluid) of about or at least about 20%, 40%, 60%, 80%, 100%, 120%, 140%, 160%, 180%, 200%, 220%, 240%, 260%, 280%, 300%, 350%, 400%, 500%, 600%, 700%, 800%, 900%, 1,000%, or more. In some embodiments, the maximal increase in weight (after which the weight of the microneedles can decrease as they dissolve), occurs after about or at least about 60, 75, 90, 105, 120, 135, 150, 165, 180, 195, 210, 225, 240, 300, 360, 420, 480, 600, 720, or more minutes.

Combinations of non-crosslinked, lightly crosslinked and extensively crosslinked microneedles 26 can be combined in a single device so as to deliver a bolus dose of an active agent e.g. or therapeutic substance(s), achieving a therapeutic plasma level, followed by controlled delivery to maintain this level. This strategy can be successfully employed whether the therapeutic substance is contained in the microneedles 26 and substrate 28 or in an attached reservoir (not shown).

In further embodiments, the substrate 28 and microneedles 26 may contain in their matrix, defined quantities of one or more water soluble excipients. Upon insertion into skin these excipients will dissolve leaving pores behind in the matrix of substrate 28 and microneedles 26. This can enhance the rate of release, which can be further controlled by changing the excipient, its concentration and/or its particle size. Suitable excipients include, but are not limited to glucose, dextrose, dextran sulfate, sodium chloride and potassium chloride or other water soluble excipients known in the art.

In use, the microneedles 26 may be inserted into the skin by gentle applied pressure or by using a specially-designed mechanical applicator applying a pre-defined force. An additional device may be used to reduce the elasticity of skin by stretching, pinching or pulling the surface of the skin so as to facilitate insertion of the microneedles 26. This latter function could be usefully combined with the function of the applicator to produce a single integrated device for insertion of a microneedle array.

Microneedles 26 composed of polymers known to form hydrogels can be manufactured by any such methods known in the art. For example, they can be prepared by a micromolding technique using a master template, such as a microneedle array made from one or more of a wide variety of materials, including for example, but not limited to, silicon, metal, and polymeric material. Master templates can be prepared by a number of methods, including, but not limited to, electrochemical etching, deep plasma etching of silicon, electroplating, wet etch processes, micromolding, microembossing, “thread-forming” methods and by the use of repetitive sequential deposition and selective x-ray irradiation of radiosensitive polymers to yield solid microneedle arrays.

Micromolds can be prepared by coating the master template with a liquid monomer or polymer which is then cured and the master template removed to leave a mold containing the detail of the master template. In the micromolding technique, a liquid monomer, with or without initiator and/or crosslinking agent is placed in the mold, which is filled by means of gravitational flow, application of vacuum or centrifugal forces, by application of pressure or by injection molding. The monomer may then be cured in the mould by means of heat or application of irradiation (for example, light, UV radiation, x-rays) and the formed microneedle array, which is an exact replicate of the master template is removed. Alternatively, a solution of a polymer with or without crosslinking agent can be placed in the mold, which is filled by means of gravitational flow, application of vacuum or centrifugal forces, by application of pressure or by injection molding. The solvent can then be evaporated to leave behind a dried microneedle array, which is an exact replicate of the master template, and can then be removed from the mold. The solvents that can be used include, but are not limited to, water, acetone, dichloromethane, ether, diethylether, ethyl acetate. Other suitable solvents will be obvious to one skilled in the art. Micromolds can also be produced without the need for master templates by, for example, micromachining methods and also other methods that will be obvious to those skilled in the art.

For example, in one embodiment, the microneedle arrays may be prepared using micromolds prepared using a method in which the shape of the desired microneedles are drilled into a suitable mold material, for example using a laser and the molds are then filled using techniques known in the art or as described herein.

Microneedles 26 composed of polymers known to form hydrogels can also be manufactured using a “self-molding” method. In this method, the polymeric material is first made into a thin film using techniques well known in the art, including for example, but not limited to, casting, extrusion and molding. The material may, or may not be crosslinked before the “self-molding” process. In this process, the thin film is placed on a previously-prepared microneedle array and heated. Plastic deformation due to gravity causes the polymeric film to deform and, upon hardening, create the desired microneedle structure.

Microneedles 26 with a hollow bore can be manufactured by using molds prepared from hollow master templates or suitably altering the micromachining methods or other methods used to prepare solid microneedles. Hollow bores can also be drilled mechanically or by laser into formed microneedles 26. Microneedles 26 which have at least one channel running down at least one outer surface from tip to substrate 28 can also be produced by suitable modification of the method used to prepare solid microneedles. Such alterations will be obvious to those skilled in the art. Channels can also be drilled mechanically or by laser into formed microneedles 26.

Microneedles 26 composed of polymers known to form hydrogels can also be manufactured using a “thread forming” method whereby a polymer solution spread on a flat surface has its surface contacted by a projection which is then moved upwards quickly forming a series of polymer “threads”, which then dry to form microneedles.

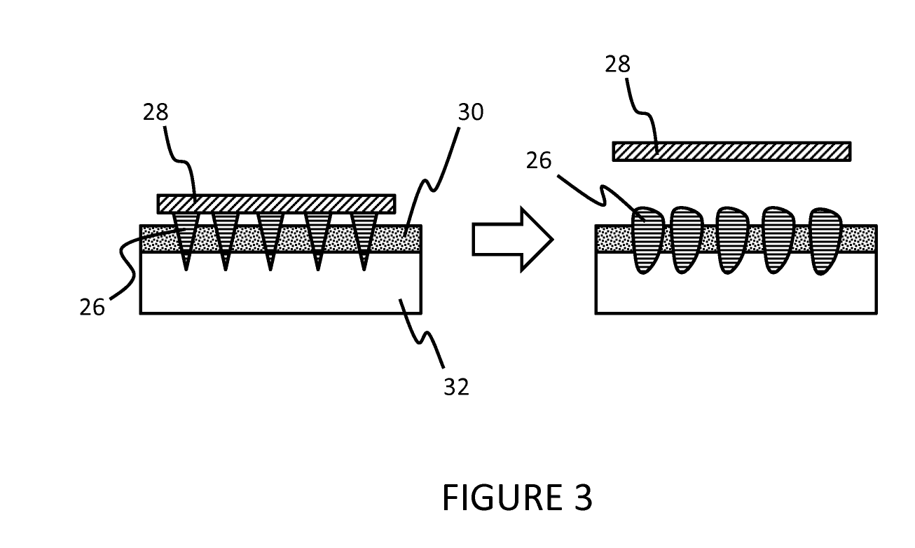

Also disclosed herein are microneedle arrays configured to allow for prolonged retention of hyaluronic acid in skin to enable sustained improvement in skin appearance. Microneedles 26 can be fabricated from mechanically-robust, yet moisture-swellable, hyaluronic acid-chitosan complexes, with substrates 28 prepared from a suitable moisture-soluble, supportive material. Once inserted into skin the microneedles 26 can imbibe skin interstitial fluid, with subsequent diffusion of such fluid to the microneedle-substrate interface 38, as illustrated in FIG. 3. This will cause separation of microneedles 26 and substrate 28, such that the microneedles 26 remain in skin post removal of the substrate 28. Complexing with chitosan can permit prolonged in skin retention, whilst removal of the substrate 28 will allow skin to reseal, thus rapidly returning skin barrier function to normal.

In some embodiments, hyaluronic acid-chitosan complexes can first be formulated. Moisture-soluble substrate materials can then be prepared. Microneedles 26 can then be prepared using a 2-step method of manufacture and evaluated for physical properties, skin insertion capabilities and skin deposition of hyaluronic acid.

In some embodiments, moisture-swellable hyaluronic acid-chitosan complexes can be prepared from aqueous blends utilizing a range of defined concentrations of stipulated molecular weights of each compound. Films can be cast and assessed for their physical properties (mechanical strength, flexibility) and swelling capabilities using standard methods. Materials that are homogenous, hard in the dry state and capable of imbibing simulated interstitial fluid and swelling can be advantageous in some embodiments. Moisture soluble substrate materials can be prepared from hyaluronic acid, with the addition of, or substation with, suitable pharma-grade polymers (e.g., carboxymethylcellulose, poly(vinylpyrrollidone) to achieve the desired performance. Some materials can possess a mechanically-robust nature and solubility in simulated interstitial fluid, and/or the ability to adhere strongly to one another. Hyaluronic acid can be used having various molecular weights. High molecular weight HA as used herein describes a HA material having a molecular weight of at least about 1.0 million Daltons (mw≧10⁶ Da or 1 MDa) to about 4.0 MDa. For example, the high molecular weight HA in the present compositions may have a molecular weight of about 2.0 MDa. In another example, the high molecular weight HA may have a molecular weight of about 2.8 MDa. Low molecular weight HA as used herein describes a HA material having a molecular weight of less than about 1.0 MDa. Low molecular weight HA can have a molecular weight of between about 200,000 Da (0.2 MDa) to less than about 1.0 MDa, for example, between about 300,000 Da (0.3 M Da) to about 750,000 Da. (0.75 MDa). In some embodiments, the hyaluronic acid component encompasses a range of hyaluronic acids having a distribution of molecular weights, such as a Gaussian distribution in some cases. As such, the molecular weight can be expressed as an average molecular weight reflecting a varying distribution of hyaluronic acid species having different molecular weights. In some embodiments, the HA can be a sodium hyaluronate and can have a molecular weight or an average molecular weight of between about 250,000 Da and about 450,000 Da, such as between about 300,000 Da and about 400,000 Da, or about 300,000 Da, 310,000 Da, 320,000 Da, 330,000 Da, 340,000 Da, 350,000 Da, 360,000 Da, 370,000 Da, 380,000 Da, 390,000 Da, or 400,000 Da. In some embodiments, the HA can have a molecular weight or an average molecular weight of between about 0.85 MDa and about 3 MDa, between about 0.85 MDa and about 1.6 mDa, between about 1.6 mDa and about 2.9 MDa, or about 0.85, 0.90, 0.95, 1.00, 1.05, 1.1, 1.15, 1.2, 1.25, 1.30, 1.35, 1.40, 1.45, 1.50, 1.55, 1.60, 1.65, 1.70, 1.75, 1.80, 1.85, 1.90, 1.95, 2.00, 2.05, 2.10, 2.15, 2.20, 2.25, 2.30, 2.35, 2.40, 2.45, 2.50, 2.55, 2.60, 2.65, 2.70, 2.75, 2.80, 2.85, 2.90, 2.95, 3.00 mDa, or any range including two of the previous values.

In some embodiments, the low molecular weight HA can make up between about 0.5% and about 50% w/w percent of the composition (e.g., the microprojection), such as between about 1% and about 30% w/w, between about 5% and about 30% w/w, between about 10% and about 25% w/w, between about 10% and about 20% w/w, or about 8%, 9%, 10%, 11%, 12%, 13%, 14%, 15%, 16%, 17%, 18%, 19%, 20%, 21%, 22%, 23%, 24%, 25%, 26%, 27%, 28%, 29%, 30% w/w of the microprojection, or a range including any two of the preceding values.

In some embodiments, the high molecular weight HA can make up between about 0.5% and about 10% w/w percent of the composition (e.g., the microprojection), such as between about 0.5% and about 3% w/w, between about 1% and about 3% w/w, between about 1% and about 2% w/w, or about 0.5%, 0.75%, 1%, 1.25%, 1.5%, 1.75%, 2%, 2.25%, 2.5%, 2.75%, 3%, 3.5%, 4%, 4.5%, 5% w/w of the microprojection, or a range including any two of the preceding values.

In some embodiments, the chitosans could include, for example, ultrapure chitosan salts and bases. Some suitable chitosans are Protasans from NovaMatrix; Sandvika, Norway. PROTASAN UP CL 113 is based on a chitosan where between 75-90 percent of the acetyl groups are removed. The cationic polymer is a highly purified and well-characterized water-soluble chloride salt. The functional properties are described by the molecular weight and the degree of deacetylation. Typically, the molecular weight for PROTASAN UP CL 113 is in the 50000-150000 g/mol range (measured as a chitosan acetate). PROTASAN UP CL 114 is based on a chitosan where more than 90 percent of the acetyl groups are removed. The cationic polymer is a highly purified and well-characterized water-soluble chloride salt. The functional properties are described by the molecular weight and the degree of deacetylation. Typically, the molecular weight for PROTASAN UP CL 114 is in the 50000-150000 g/mol range (measured as a chitosan acetate). PROTASAN UP CL 213 is based on a chitosan where between 75-90 percent of the acetyl groups are removed. The cationic polymer is a highly purified and well-characterized water-soluble chloride salt. The functional properties are described by the molecular weight and the degree of deacetylation. Typically, the molecular weight for PROTASAN UP CL 213 is in the 150000-400000 g/mol range (measured as a chitosan acetate). PROTASAN UP CL 214 is based on a chitosan where more than 90 percent of the acetyl groups are removed. The cationic polymer is a highly purified and well-characterized water-soluble chloride salt. The functional properties are described by the molecular weight and the degree of deacetylation. Typically, the molecular weight for PROTASAN UP CL 214 is in the 150000-400000 g/mol range (measured as a chitosan acetate). PROTASAN UP G 113 is based on a chitosan where between 75-90 percent of the acetyl groups are removed. The cationic polymer is a highly purified and well-characterized water-soluble chloride salt. The functional properties are described by the molecular weight and the degree of deacetylation. Typically, the molecular weight for PROTASAN UP G 113 is in the 50000-150000 g/mol range (measured as a chitosan acetate). PROTASAN UP G 114 is based on a chitosan where more than 90 percent of the acetyl groups are removed. The cationic polymer is a highly purified and well-characterized water-soluble chloride salt. The functional properties are described by the molecular weight and the degree of deacetylation. Typically, the molecular weight for PROTASAN UP G 114 is in the 50000-150000 g/mol range (measured as a chitosan acetate). PROTASAN UP G 213 is based on a chitosan where between 75-90 percent of the acetyl groups are removed. The cationic polymer is a highly purified and well-characterized water-soluble chitosan glutamate. The functional properties are described by the molecular weight and the degree of deacetylation. Typically, the molecular weight for PROTASAN UP G 213 is in the 150000-600000 g/mol range (measured as a chitosan acetate). PROTASAN UP G 214 is based on a chitosan where more than 90 percent of the acetyl groups are removed. The cationic polymer is a highly purified and well-characterized water-soluble chloride salt. The functional properties are described by the molecular weight and the degree of deacetylation. Typically, the molecular weight for PROTASAN UP G 214 is in the 150000-400000 g/mol range (measured as a chitosan acetate). In some embodiments, the chitosan can make up between about 0.5% and about 50% w/w percent of the composition (e.g., the microprojection), such as between about 1% and about 25% w/w, between about 1% and about 10% w/w, between about 1% and about 5% w/w, between about 2% and about 3% w/w, or about 1%, 1.5%, 2%, 2.5%, 3%, 3.5%, 4%, 4.5%, 5%, 5.5%, 6%, 6.5%, 7%, 7.5%, 8%, 8.5%, 9%, 9.5%, or 10% w/w of the microprojection.

In some embodiments, HA can be complexed with a suitable crosslinking agent. The crosslinking agent may be any agent known to be suitable for crosslinking polysaccharides and their derivatives via their hydroxyl groups. Suitable crosslinking agents include, but are not limited to, 1,4-butanediol diglycidyl ether (or 1,4-bis(2,3-epoxypropoxy)butane or 1,4-bisglycidyloxybutane, all of which are commonly known as BDDE), 1,2-bis(2,3-epoxypropoxy)ethylene and 1-(2,3-epoxypropyl)-2,3-epoxycyclohexane. The use of more than one crosslinking agent or a different crosslinking agent is not excluded from the scope of the present disclosure. The step of crosslinking may be carried out using any means known to those of ordinary skill in the art. Those skilled in the art appreciate how to optimize conditions of crosslinking according to the nature of the HA, and how to carry out crosslinking to an optimized degree. Degree of crosslinking for purposes of the present disclosure is defined as the percent weight ratio of the crosslinking agent to HA-monomeric units within the crosslinked portion of the HA based composition. It is measured by the weight ratio of HA monomers to crosslinker (HA monomers:crosslinker). In some embodiments, the degree of crosslinking in the HA component of the present compositions is at least about 2% and is up to about 20%. In other embodiments, the degree of crosslinking is greater than 5%, for example, is about 6% to about 8%. In some embodiments, the degree of crosslinking is between about 4% to about 12%. In some embodiments, the degree of crosslinking is less than about 6%, for example, is less than about 5%. In some embodiments, the HA component is capable of absorbing at least about one time its weight in water. When neutralized and swollen, the crosslinked HA component and water absorbed by the crosslinked HA component is in a weight ratio of about 1:1. The resulting hydrated HA-based gels have a characteristic of being highly cohesive.

Materials including those described elsewhere herein can form microneedle arrays using a micromolding technique. Laser-engineered silicone elastomer molds of a range of geometries can be utilized to form microneedles using a 2-step process. Aqueous blends of hyaluronic acid-chitosan can be initially cast into the molds, allowed to dry and then the moisture-swellable blend can be added to form the baseplate. Upon demolding, microneedle arrays can be studied using light and scanning electron microscopy.

In some embodiments, molding can occur by adding the HA formulation or formulations (e.g., an amount of a high molecular weight HA and/or an amount of a low molecular weight HA) and/or a chitosan formulation to the molds in a primary casting step. Following centrifugation (e.g., about 3500 rpm for about 15 minutes in some embodiments), the needle casting can be allowed to dry at a particular temperature (e.g., room temperature) for a specified time (e.g, about 1, 1.5, or 2 hours) before an amount of a polymer, such as PVP for example, is then added. Another optional centrifugation step can occur (e.g., about 3500 rpm for an additional 5 minutes), and the molds are then allowed to dry overnight.

In any of the above methods, substances to be incorporated into the microneedles 26 themselves (e.g., active therapeutic agents, pore forming agents, enzymes etc.) can be added into the liquid monomer or polymer solution during the manufacturing process. Alternatively, such substances can be imbibed from their solution state in a solution used to swell the formed microneedle arrays and dried thereafter or the formed arrays can be dipped into a solution containing the agent of interest or sprayed with a solution containing the agent of interest. Solvents used to make these solutions include water, acetone, dichloromethane, ether, diethylether, ethyl acetate. Other suitable solvents will be obvious to those skilled in the art, as will the processes used to dry the microneedle arrays. If the microneedles 26 and/or substrate 28 are to be made adhesive, the formed arrays can be dipped into a solution containing an adhesive agent or sprayed with a solution containing an adhesive agent. The adhesive agents used can be a pressure sensitive adhesive or a bioadhesive. These substances are well known and will be obvious to those skilled in the art.

The substrate 28 on which the microneedles 26 are formed can be varied in thickness by suitable modification of the method of manufacture, including, for example, but not limited to increasing the quantity of liquid monomer or polymer solution used in the manufacturing process. In this way the barrier to diffusion/transport of therapeutic active agents can be controlled so as to achieve, for example rapid delivery or sustained release. Where therapeutic active agent(s) is/are to be contained within the matrix of the microneedles 26 and substrate 28, the thickness of the substrate 28 can usefully be increased so as it functions as a fully integrated reservoir.

Crosslinks may be physical or chemical and intermolecular or intramolecular. Methods for crosslinking polymers are well known in the art. Crosslinking is the process whereby adjacent polymer chains, or adjacent sections of the same polymer chain, are linked together, preventing movement away from each other. Physical crosslinking occurs due to entanglements or other physical interaction. With chemical crosslinking, functional groups are reacted to yield chemical bonds. Such bonds can be directly between functional groups on the polymer chains or a crosslinking agent can be used to link the chains together. Such an agent could possess at least two functional groups capable of reacting with groups on the polymer chains. Crosslinking prevents polymer dissolution, but may allow a polymer system to imbibe fluid and swell to many times its original size.

The microneedle array can be used for cosmetic or medical applications. In some embodiments, the microneedle array can include one, two, or more water-soluble pharmaceutical-grade polymers including those that can dissolve or degrade in vivo, including polysaccharides such as hyaluronic acid, chondroitin sulfate, glycogen, dextrin, dextran, dextran sulfate, hydroxypropyl methylcellulose, alginic acid, chitin, chitosan, and pullulan; proteins such as collagen, gelatin, and hydrolysates thereof; synthetic high polymers such as polyvinyl alcohol, polyvinyl pyrrolidone (PVP), polyacrylic acid, and carboxyvinyl polymer; carboxymethylcellulose, and the like. In some embodiments, not to be limited by theory, addition of the one or more polymers can provide devices, such as microneedle arrays with increased strength and render them mechanically more robust, while still maintaining flexibility. In some embodiments, the polymer can have a molecular weight of between about 100 kDa and about 500 kDa, such as about, at least about, or no more than about 100, 150, 200, 250, 300, 350, 360, 370, 380, 390, 400, 450, or 500 kDa. In some embodiments, PVP having a molecular mass range of from about 25 kDa to about 60 kDa can be used such that renal clearance of the polymer is improved, and toxicity may be reduced. In some embodiments, the polymer, e.g., PVP, can have a relatively low molecular weight, e.g., a molecular weight of about, or no more than about 25 kDa, 30 kDa, 35 kDa, 40 kDa, 45 kDa, 50 kDa, 55 kDa, 56 kDa, 57 kDa, 58 kDa, 59 kDa, 60 kda, or 65 kDa, or a range incorporating any two of the previous values. In some embodiments, the polymer can make up between about 1% and about 50% w/w percent of the composition (e.g., the microprojection), such as between about 1% and about 25% w/w, between about 5% and about 15% w/w, or about 5%, 6%, 7%, 8%, 9%, 10%, 11%, 12%, 13%, 14%, 15%, 20%, or 25% w/w of the microprojection.

In some embodiments, the microneedles 26 can include a proteoglycan. Proteoglycan is a general term for molecules in which one or more glycosaminoglycans are covalently linked to a core protein. The type of proteoglycan used can include, for example, chondroitin sulfate proteoglycan, dermatan sulfate proteoglycan, heparan sulfate proteoglycan, and keratan sulfate proteoglycan. Specific examples include aggrecan, versican, neurocan, brevican, decorin, biglycan, serglycin, perlecan, syndecan, glypican, lumican, keratocan, etc.

In some embodiments, the microneedles are configured to deliver a payload into the tissue of HA of about 0.5 to about 5 mg/cm², about 0.6 to about 3.1 mg/cm², or about or at least about 0.5, 0.6, 0.7, 0.8, 0.9, 1.0, 1.5, 2.0, 2.5, 3.0, 3.5 mg/cm², or more.

Various microneedle features that can be used or modified for use with the invention can be found, for example, in U.S. Pat. No. 6,256,533 to Yuzhakov et al., U.S. Pub. No. 2009/0082719 to Friden, U.S. Pub. No. 2010/0256064 to Woolfson et al., U.S. Pub. No. 2013/0012882 A1 to Quan et al., PCT Pub. No. WO 2012/131623 A3 to Hirt et al., PCT Pub. No. WO 2008/053481 A1 to Guy et al., and EP Pub. No. 2653186 A2 to Jung et al., all of which are hereby incorporated by reference in their entireties.

### Indications

The compositions help treat or prevent any number of conditions, including dermatologic conditions such as severe skin dryness, dullness, loss of elasticity, lack of radiance, exaggerated lines and wrinkles, spider vessels or red blotchiness. In some embodiments, “marionette” lines, smile lines, deep nasolabial fold lines, crow's feet, fine lines/wrinkles, vertical lines between the eyebrows, horizontal forehead lines, sagging thin/frail skin, skin redness and dullness may be improved using compositions as described herein. The compositions can also be used in the prevention and treatment of: photodamaged skin, the appearance of fine lines and wrinkles, hyperpigmentation, age spots, and aged skin. The disclosed composition can also increasing the flexibility of the stratum corneum, increasing the content of collagen and/or glycosaminoglycans in skin, increasing moisture in skin, decreasing transcutaneous water loss, and generally increasing the quality of skin. The disclosed composition also provides topical formulations effective in promoting a healthy scalp, and thereby useful in the prevention of hair loss, and as a treatment before and after hair transplant surgical procedures.

It is contemplated that various combinations or subcombinations of the specific features and aspects of the embodiments disclosed above may be made and still fall within one or more of the inventions. Further, the disclosure herein of any particular feature, aspect, method, property, characteristic, quality, attribute, element, or the like in connection with an embodiment can be used in all other embodiments set forth herein. Accordingly, it should be understood that various features and aspects of the disclosed embodiments can be combined with or substituted for one another in order to form varying modes of the disclosed inventions. Thus, it is intended that the scope of the present inventions herein disclosed should not be limited by the particular disclosed embodiments described above. Moreover, while the invention is susceptible to various modifications, and alternative forms, specific examples thereof have been shown in the drawings and are herein described in detail. It should be understood, however, that the invention is not to be limited to the particular forms or methods disclosed, but to the contrary, the invention is to cover all modifications, equivalents, and alternatives falling within the spirit and scope of the various embodiments described and the appended claims. Any methods disclosed herein need not be performed in the order recited. The methods disclosed herein include certain actions taken by a practitioner; however, they can also include any third-party instruction of those actions, either expressly or by implication. For example, actions such as “administering a hyaluronic acid formulation” include “instructing the administration of a hyaluronic acid formulation.” The ranges disclosed herein also encompass any and all overlap, sub-ranges, and combinations thereof. Language such as “up to,” “at least,” “greater than,” “less than,” “between,” and the like includes the number recited. Numbers preceded by a term such as “about” or “approximately” include the recited numbers. For example, “about 3 mm” includes “3 mm.”

### EXAMPLES

Specific embodiments will be described with reference to the following examples, which should be regarded in an illustrative rather than a restrictive sense.

### Example 1

### Two-Step Casting Methodology

Microneedle arrays were prepared using a two-step casting methodology, one embodiment of which is described by the present example. A silicone sheet was laser cut to create a mold of the microneedle array. The laser-cut silicone sheet was then glued to the bottom of a silicone well. Microneedle casting material was poured into the silicone well, filling the recesses that had been laser cut into the silicone sheet. In at least one embodiment, the microneedle casting material comprised 1.5% w/w Hyaluronsan HA-LQSH, 2.5% Protasan UP CL 213, 10% w/w 58 kDa PVP, and 76% 10 mM potassium phosphate buffer, pH 4.6. After pouring the microneedle casting material into the silicone well, the silicone well was subjected to centrifugation (3600 rpm for 15 minutes) to compress the microneedle casting material into the laser-cut recesses of the silicone sheet. Substrate casting material was then poured into the silicone well and layered on top of the microneedle casting material. In some embodiments, the substrate casting material comprised 40% w/w PVP dissolved in water, the PVP having a molecular weight of 58 kDa. The silicone well was again subjected to centrifugation (3600 rpm for 5 minutes). The casting was then dried overnight at room temperature.

### Example 2

### Casting Formulations

The following formulations are non-limiting examples that can be used to cast microneedle arrays.

10% w/w PVP 360 kDa; 2.5% w/w Protasan UP CL 213; 25% w/w Hyabest
10% w/w PVP 58 kDa; 2.5% w/w Protasan UP CL 213; 12.5% w/w Hyabest
10% w/w PVP 58 kDa; 2.5% w/w Protasan UP CL 213; 10% w/w Hyabest
10% w/w PVP 58 kDa; 2.5% w/w Protasan UP CL 213; 1.5% w/w Hyaluronsan HA-LQ
10% w/w PVP 58 kDa; 2.5% w/w Protasan UP CL 213; 1.5% w/w Hyaluronsan HA-LQSH

The above listed casting formulations were prepared in 10 mM potassium phosphate buffer, pH 4.6. PVP 58 kDa was obtained from Ashland Inc. (Covington, Ky., USA). PVP 360 kDa was obtained from Sigma-Aldrich (Steinheim, Germany). Hyabest (Sodium Hyaluronate), Hyaluronsan HA-LQ, and Hyaluronsan HA-LQSH were obtained from Kewpie Corporation (Tokyo, Japan).

### Example 3

### Microneedle Swelling

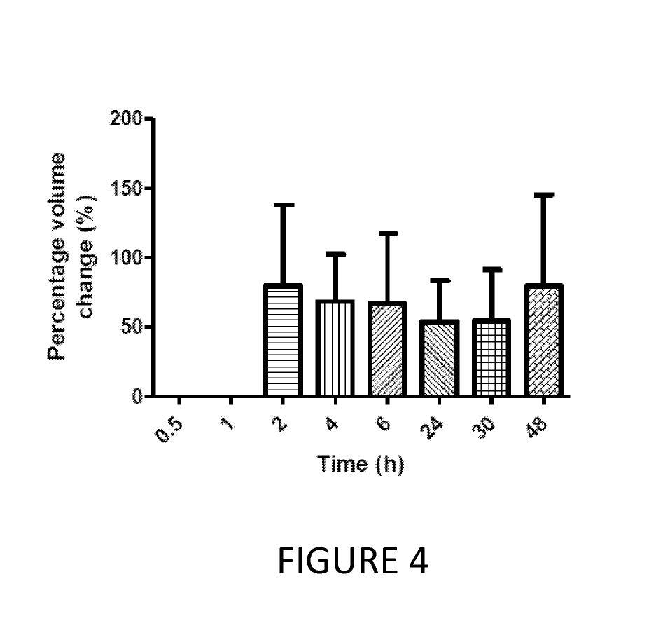

Skin penetration and swelling of microneedle preparations was assessed using optical coherence tomography (OCT) and a porcine skin model. Full thickness, shaved, neonatal porcine skin was employed as the skin model. 500 μm-thick sections of skin were placed dermal side down onto an absorbent wound dressing. Microneedles were inserted manually. Real time high resolution imaging of the upper skin layers was performed using a swept-source Fourier domain OCT system at a wavelength of 1305.0+/−15.0 nm. Images were analyzed to determine volumetric changes to microneedles over time. FIG. 4 depicts the volumetric change over time for microneedles cast from a formulation including 1.5% w/w Hyaluronsan HA-LQSH, 2.5% w/w Protasan UP CL 213, 10% w/w 58 kDa PVP, 76% 10 mM potassium phosphate buffer, pH 4.6. As illustrated and described, the formulations demonstrated superior and unexpected significant swelling capabilities.

### Example 4

### Microneedle Swelling

Microneedle swelling was assessed for different formulations of microneedle materials. Microneedle arrays were made by castings using the different formulations. The dry weight of the microneedle array was recorded. Microneedle arrays were then submerged in phosphate buffered saline (PBS), pH 7.4, at room temperature. The arrays were removed from PBS at specific time points, blotted to remove excess PBS, and weighed. Percent swelling was calculated by subtracting from the mass at time “t” the dry mass (i.e., t=0) and then dividing by the dry mass. As illustrated and described, the formulations demonstrated superior and unexpected significant swelling capabilities.

Formulation T29 comprises 10% w/w PVP having an average molecular weight of 360 kDa, 2.5% w/w Protasan UP CL 213, 25% w/w Hyabest, 62.5% 1.0 mM potassium phosphate buffer, pH 4.6.

Formulation T32 comprises 10% w/w PVP having an average molecular weight of 58 kDa, 2.5% w/w Protasan UP CL 213, 10% Hyabest, 77.5% 10 mM potassium phosphate buffer, pH 4.6.

Formulation T45 comprises 10% w/w PVP having an average molecular weight of 58 kDa, 2.5% w/w Protasan UP CL 213, 1.5% Hyaluronsan HA-LQ, 76% 10 mM potassium phosphate buffer, pH 4.6.

Formulation T46 comprises 10% w/w PVP having an average molecular weight of 58 kDa, 2.5% w/w Protasan UP CL 213, 1.5% Hyaluronsan HA-LQSH, 76% 10 mM potassium phosphate buffer, pH 4.6.

What is claimed is:

1. A composition of matter comprising:
between about 5% and about 15% by weight polyvinylpyrrolidone;
at least 1% by weight low-molecular weight sodium hyaluronate, the low-molecular weight sodium hyaluronate having a molecular weight between 150 kDa and 400 kDa; and
between 1% and 2% by weight high-molecular weight sodium hyaluronate, the
high-molecular weight sodium hyaluronate having a molecular weight between 1 MDa and 2 MDa.

2. The composition of matter of claim 1 wherein the polyvinylpyrrolidone has a molecular weight between 20 kDa and 100 kDa.

3. A device for delivering an agent to the skin, the device comprising:
a substrate having a top face; and
a plurality of microneedles extending from the top face of the substrate, the microneedles being made from a material comprising between 5% and 15% by weight polyvinylpyrrolidone and at least 1% by weight sodium hyaluronate having a molecular weight between 150 kDa and 400 kDa.

4. The device of claim 3 wherein the microneedles have a height in the range of 100-1000 μm.

5. The device of claim 3 wherein the microneedles have an interspacing between 50-1000 μM.

6. The device of claim 3 wherein the microneedles have a density in the range of 50-5000 microneedles per cm².

7. The device of claim 3 wherein the microneedles have a shape selected from the group consisting of: conical, pyramidal, elliptical, oval, and cylindrical.

8. The device of claim 3 wherein the microneedles are made of a material comprising hyaluronic acid, or derivatives of hyaluronic acid, that have been crosslinked with a cationic agent.

9. The device of claim 3 wherein the substrate is made of a material comprising between 20% and 50% polyvinylpyrrolidone.

10. The device of claim 9 wherein the polyvinylpyrrolidone has a molecular weight between 20 kDa and 100 kDa.

11. A method of making a device having moisture-swellable microneedles, the method comprising:
providing a mold;
placing a first material into the mold, the first material comprising:
between 5% and 15% by weight polyvinylpyrrolidone;
at least 1% by weight low-molecular weight sodium hyaluronate, the low-molecular weight sodium hyaluronate having a molecular weight between 150 kDa and 400 kDa; and
between 1% and 2% by weight high-molecular weight sodium hyaluronate, the high-molecular weight sodium hyaluronate having a molecular weight between 1 MDa and 2 MDa;
placing a second material into the mold, the second material being layered on top of the first material;
allowing the first and second material to dry; and
removing the first and second material from the mold.

12. The method of claim 11 wherein the first material comprises:
between 5% and 10% by weight polyvinylpyrrolidone;
at least 1% by weight low-molecular weight sodium hyaluronate, the low-molecular weight sodium hyaluronate having a molecular weight between 150 kDa and 400 kDa; and
between 1% and 2% by weight high-molecular weight sodium hyaluronate, the
high-molecular weight sodium hyaluronate having a molecular weight between 1 MDa and 2 MDa.

13. The method of claim 11 wherein the second material comprises between 20% and 50% by weight polyvinylpyrrolidone.

14. The method of claim 13 wherein the polyvinylpyrrolidone has a molecular weight between 20 kDa and 100 kDa.

15. A device comprising a first element consisting of an array of microprojections and a second element comprising a supportive substrate upon which the microprojections are formed substantially perpendicular to the substrate surface.

16. A device according to claim 15 wherein the microprojections have heights in the range of about 100-1000 μm.

17. A device according to claim 15 wherein the microprojections have widths at base in the range of about 50-500 μm.

18. A device according to claim 15, wherein the density of the microprojections on the supportive substrate is in the range of about 50-5000 microprojections per cm².

19. A device according to claim 15, wherein the microprojections are conical in shape.

20. A device according to claim 15, wherein the microprojections are cylindrical in shape.

21. A device according to claim 15, wherein the microprojections are pyramidal in shape.

22. A device according to claim 15, wherein the microprojections are comprised of hyaluronic acid or derivatives thereof.

23. A device according to claim 15, wherein the hyaluronic acid has an average molecular weight in the range of about 100,000 Daltons to 2,000,000 Daltons.

24. A device according to claim 15, wherein the microprojections are comprised of hyaluronic acid or derivatives thereof crosslinked with a cationic agent.

25. A device according to claim 24, wherein the cationic agent comprises chitosan or a derivative thereof.

26. A device according to claim 15, further comprising poly(vinylpyrrolidone), poly(vinylalcohol), a cellulose derivative or other water soluble biocompatible polymer configured to imbue mechanical strength sufficient to allow skin penetration upon application by the human hand or a suitable mechanical applicator device.

27. A device according to claim 15, wherein the microprojections swell in skin interstitial fluid upon skin insertion.

28. A device according to claim 15, wherein the microprojections dissolve in skin interstitial fluid upon skin insertion.

29. A device according to the claim 15, where the supportive substrate is water soluble and dissolves upon skin insertion of the microprojections within about 15 minutes to about 6 hours.

30. A device according to claim 15, wherein single or repeated use causes a noticeable increase in skin volume at the site of application.

31. A device according to claim 15, wherein single or repeated use causes a noticeable reduction in the appearance of wrinkles, fine lines, stretch marks or acne scars at the site of application.

32. A device according to claim 15, wherein the device is attached to a third element comprising an adhesive layer al lowing skin retention and a protective water-insoluble occlusive layer.

33. A device according to claim 32, wherein a fourth element is introduced comprising electrodes and a source of direct or alternating electrical current.

34. A device according to claim 33, wherein application of an electrical current enhances the rate of hyaluronic acid deposition in the viable skin layers or accelerates microprojection dissolution and/or swelling in skin or accelerates dissolution of the supportive substrate on the skin surface.
